(19) United States
(12) Patent Application Publication
(10) Pub. No.: US 2016/0089035 A1
(43) Pub. Date: Mar. 31, 2016

(54) Method of setting the blood flow through the bloodstream of a live being and device for an indicator application

(71) Applicant: VSB - Technická univerzita Ostrava, Ostrava Poruba (CZ)

(72) Inventors: Frantisek Jurek, Ostrava (CZ); Martin Augustynek, Ostrava - Poruba (CZ); Marek Penhaker, Ostrava (CZ); Stepán Ozana, Ostrava Zabreh (CZ)

(21) Appl. No.: 14/619,531

(22) Filed: Feb. 11, 2015

(30) Foreign Application Priority Data
Sep. 25, 2014 (CZ) PV 2014-661

(51) Int. Cl. A61B 5/026; A61B 5/145

(52) U.S. Cl. CPC A61B 5/026; A61B 5/14532

(57) ABSTRACT

Blood flow rate in the bloodstream of a live being is measured using glucose as an indicator continuously supplied to the bloodstream. A reference sample is taken in a selected of measured segment of the bloodstream and then, an indicator of known concentration and known flow is added to the bloodstream, while—with a time delay—there is performed the taking of at least one blood sample from a measuring point, located downstream, while the resulting flow rate through the measured segment is set on the basis of a functional relationship.

### FIELD OF THE INVENTION

The invention relates to the method of setting the blood flow through the bloodstream in a live being body and the device for its setting, which is used for application of an indicator of known concentration, in this case the glucose.

### BACKGROUND OF THE INVENTION

Cardiac output setting is an important piece of information for doctors in many medicine cases. The heart output is defined as a specific volume of blood per time unit (flow rate), by which heart supplies all and any body organs and tissues through circulation of blood. Cardiac output or minute volume (CO—cardiac output, MV—minute volume) of an adult man is approximately 5.5 l.min⁻¹. Cardiac output correlates with oxygen concentration in cells, which is an interesting but hard-to-measure figure. As CO is the function of the body, it is usually assessed using the cardiac index (CI—cardiac index) related to 1 m² of body surface (CI=CO/BSA, where BSA=body surface area in m². Standard values of CI are between 2.8 and 3.6 liter.m⁻²). Knowledge of the CO (CI) value allows calculations of other parameters of large and small circulation, like vascular resistance or work of left and right ventricle. It is necessary to state that cardiac output measuring is just an example of the most frequent measuring of blood flow, but there are also other segments of bloodstream, where it is suitable to measure the same physical value, i.e. flow rate. Methods of CO measuring can be classified from several points of view:
Indirect (non-invasive) methods, like ultrasound, radionuclide, impedance; even the Fick's method is included.
Direct (invasive) methods, like electromagnetic flow meter, ventriculography.
Dilution (invasive) methods, based on detection and assessment of the indicator added to the blood flow and mixed with blood and in the location of its detection which is in a suitable distance downstream. The indicator concentration is recorded and assessed (dilution curve).
Depending on the purpose of use it is possible to select the method for a one-off measuring, e.g. pigment dilution to set the flow through specific heart structures or its large vessels with valves for the cardio-surgery needs, or a method allowing repeated measuring or monitoring in the course of treatment and medication mainly in intensive medicine. For this purpose there are used orientation ultrasound methods and dilution methods, most frequently the thermo-dilution ones. There were also performed experiments with indicators affecting the blood conductivity.

The indicator that may be added to the bloodstream is subject to quite strict conditions. The body must tolerate well the indicator, the indicator must not be toxic, it must be easy-to-mix with blood. It must not disappear from the measured segment of the bloodstream, it must be easy-to-measure, ideally with linear dependence of the measuring result on real concentration and its re-circulation should be minimal or none. Pigment/dye and heat are usually used as indicators, while pigment dilution and thermo-dilution in various modifications are the most commonly used measuring methods.

The Fick's method allows calculation of the cardiac output from the difference of mixed venous blood and arterial blood saturation with oxygen while knowing the quantity of oxygen accepted by the organism (Adolf Fick, 1870). The Fick's method is naturally non-toxic, it uses oxygen as indicator and there is measured the average cardiac output.

The thermo dilution method uses heat as an indicator. The medium is usually the physiological solution or 5% solution of glucose cooled down to approximately 4° C. in water, but the indicator may also be some heated specific volume of flowing blood and there measured the course of temperature change downstream. For CO measuring, the cooled solution or heated blood part is mixed in the right atrium of heart with blood flow and the course of mixture temperature is recorded in pulmonary artery. Measuring with cooled indicator is performed using the Swan-Ganz catheter, with high-speed response temperature sensor (thermistor) placed in its end. The use of Swan-Ganz catheter allows measuring of other parameters (invasive measuring of blood pressure, venous blood saturation with oxygen, etc.). The catheter contains two channels. One of them comes to a proximal hole and it serves for cooled indicator supply. The orifice is placed in front of the right atrium of the heart. The other channel of the catheter comes to a distal hole which is placed in the pulmonary artery and it serves for direct measuring of pressure in its placement location. The distal end of the S-G catheter contains the distal hole and thermistor as well as an inflatable balloon. There is measured the temperature change in time. The specific heat capacity of blood depends on haematocrit, which is the share of erythrocytes volume and complete blood volume. For CO measuring there is usually used quick provision of 10 ml of indicator. There is recorded the temperature change of blood flow in pulmonary artery and there is assessed the area of such a dilution curve obtained. By combination of the curve integral and volume of power taken by the blood cooling, the measuring provides data on flow of the heart pumping the blood to the small circulation system (CO in l.min⁻¹).

$$CO = 60 \cdot k \cdot \frac{{ρ}_{i} {c}_{i}}{{ρ}_{b} {c}_{b}} \cdot \frac{{V}_{i} ( {ϑ}_{b} - {ϑ}_{i} )}{\frac{1}{{t}_{m}} {\int }_{0}^{{t}_{m}} ( {ϑ}_{b} - ϑ ( t ) ) d t}$$

where:
CO [l·min⁻¹] is the cardiac output per minute
ν[° C.] is the temperature in the place of measurement
ν_{b}, νᵢ [° C.] is the blood and indicator temperature
tₘ [s] is the total time of measuring
ρ_{b}, ρᵢ [kg·m⁻³] is the blood and indicator density
c_{b}, cᵢ [J·kg⁻¹·K⁻¹] is the specific thermal capacity of blood and indicator
k [−] is the correction factor of the catheter

The measuring is repeated 3 to 6 times and then the average value is calculated. That indicates that the range of values measured by thermo dilution is large. When using the blood column heating it is necessary to set the limitation for heating so as to avoid tissue damage or blood coagulation. That is why the change of blood temperature by heating is very small and easily interchangeable with the thermo-physiological changes of the blood temperature. That is why the measuring must be repeated as well and average results calculated.

The problems in measuring using the dilution method leading to extensive dispersion of measured CO values are given e.g. by physiological oscillations of basic temperature in pulmonary artery, by the fact that the thermal exchange space is not equal to the intravascular space with its contents. The measuring is influenced by placement of the temperature sensor in blood flow in the pulmonary artery (in the centre of the flow, close to the pulmonary artery wall), there is also the influence of the dead space of application channel of the catheter. Another problem is the handling of the indicator volume (with thermal energy volume) which can be solved by a complex application technical device. The temperature sensor parameters change in case of long-term placement of the catheter in the pulmonary artery.

The method of dye dilution is based on Stewart-Hamilton equation:

$$CO = \frac{{V}_{i}}{{\int }_{0}^{∞} {c}_{i} ( t ) d t}$$

A dye is used as an indicator—usually the indo-cyanide green (cardio green) diluted with the NaCl solution. The indicator is not harmful for the organism, it is decomposed by kidneys. Several grams of the dye are injected by the pulmonary catheter to the pulmonary artery. Blood from brachial or femoral artery is taken by uniform suction to the absorption photometer flask. The method is highly exact and it is used as a reference method. It allows measuring and makes possible measuring of an endocardial shunt. But it cannot be repeated in short time intervals as the dye accumulates in the bloodstream. It requires quite complicated calibration of the absorption spectrometer and calibration measuring with blood of the examined patient.

The problems of dye dilution include instability of prepared solution with dye indicator, non-linearity of measuring set by dye relation to erythrocytes and movement of the absorption spectrum, necessity of exact and linear blood sucking through the measuring flask, necessity of higher lumen of the catheter and presence of significant re-circulation wave on the dilution curve that must be solved on mathematic level. The method cannot be used for the acute medicine needs.

One of the disadvantages of the above stated substances as indicators for dilution methods in bloodstream is their distribution in blood, which is a suspension of mainly erythrocytes and blood plasma. The share of erythrocytes volume in full blood is set by the haematocrit and its value is approximately 50%. The distribution respectively mixing of added substance depends on many physical factors. The heat transfer from cold indicator is different for plasma, it is different for the erythrocytes mass and it is different for the vascular wall, surrounding the moving blood column. The added colour bodies disperse in the plasma space only, not inside the erythrocytes, but they fix to their surface. That is it is necessary to perform a special calibration measuring the dye indicator behaviour in relation to the blood of the particular patient in given location and given time.

That is why there was searched a substance meeting all and any of the above stated general requirements towards the indicator to be used in bloodstream, but also a substance that would behave in such a way so as to mix in the complete blood volume, i.e. not to disperse only in the water phase (plasma) but even in the internal space of erythrocytes. The reason for a new indicator searching is simple. The cardiac output as well as the blood flow in another segment of the bloodstream is an item describing the complete (full) blood flow. Any individual calibration and average-out constant used in the calculation makes the whole measuring more complicated and imprecise. That is why we searched for a substance behaving in the blood in such a way so as its resulting concentration in blood after mixing is set by a simple mathematic relation applicable to blood regardless its haematocrit and so as it is metabolised in patient's body and so the re-circulation is postponed. Glucose was discovered to be such a substance.

The methods of cardiac output setting as known by now are always connected with some limitation, imprecision or complexity.

The application U.S. Pat. No. 5,928,155 describes a device, a system and a method for setting the cardiac output of patient's cardiovascular system by dilution method with various indicators.

The whole system consists of a multi-channel catheter and control unit for measuring regulation and assessment and so it is able to set the heart output value quickly and without any negative influences on human organism. The catheter is introduced to the cardiovascular system percutaneously via the jugular or alternatively via the femoral vein in the heart flow direction and via the heart up to the pulmonary artery. The catheter contains a ball that is partially inflated and the blood flow pulls the catheter tip up to the target location. The target location is identified by the pulmonary blood pressure curve established using an open channel in the end of the catheter with connection to the electromanometer or by photometric setting of the of the catheter by light from the optical channel. The catheter introduction is simplified by the blood flow direction. The blood flow course from the pulmonary artery is shown in the monitor. The catheter may also include a thermal sensor for exact temperatures profile recording or optical sensors for establishment of pH or ammonia concentration.

The catheter consists of two optical channels with optical cables for pH measuring in the surroundings of the catheter tip and for measuring of indication substance concentration and also of a channel for indication substance intake into the bloodstream from the indication substance solution tank. There is another channel placed in the centre of the catheter and the channel is used for blood pressure setting. Another channel houses two electric sensors connected to the thermistor and designed for temperature measuring. In another channel of the catheter, designed for gas intake (usually CO2), gas is taken to the stabilisation balloon. The catheter body contains spots for application of medicaments into the bloodstream.

The catheter is 2,2 mm in diameter and it is 1 m long. It is made of bio-compatible and non-invasive material. The indication substance is taken to the catheter via the supply connector in the end of the catheter. The supply catheter in operation connection is connected to the control unit regulating the indication substance dosing from the solution tank. As stated above, the catheter is construed as a multi-channel one, wherein the communication between individual channels is arranged by the control unit. Outputs lead from the individual channels of the connection connector to the control unit arranging communication between them and regulating them in the course of measuring.

The control unit is controlled by a micro-processor and the whole process does not require high volume of human work for cardiac output measuring, wherein many cardiovascular parameters are available at the device display or saved on digital carriers. The control unit also monitors the indication solution concentration in blood so as to be able to assess the concentration corresponding to homeostasis. The indication solution concentration in blood is in balance with the metabolic activity of the patient's body. The homeostasis condition is indicated by a stable value of indication solution concentration.

The system is able to set the cardiac output value quickly and without any negative effects on human organism. The speed of measuring is affected by the indication solution selection. Indication solutions suitable for this method include: solution of ammonia, heparin, ethanol, carbon dioxide, glucose or anaesthetic agents
The ammonia solution as the indication solution contains—for the measuring purposes—gaseous ammonia NH₃ and its ionised form NH₄⁺. The ammonia solution was tested on animals and it was established that it did not have any influence on haematocrit (HCP) and that is why it is suitable as an indication substance.
The carbon dioxide solution releases carbon dioxide in the form of gas. The CO₂ is inert and natural gas for a body. When using the gas as an indication substance we need to measure even the following parameters:
   Partial pressure of CO₂ in blood plasma,
   pH of blood in selected area of measuring,
   Blood temperature in selected area of measuring,
   Value of haematocrit, based on which the haemoglobin value can be calculated,
   Oxygen saturation of mixed venous blood in bloodstream.
The glucose solution and its concentration is measured:
   On the basis of oxygen concentration decrease (pO₂). Oxygen in combination with glucose and glucose—oxidase enzyme melt according to the following oxidase-catalysed formula:

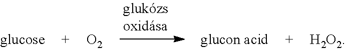

Amperometrically by a graphite electrode with incorporated ferrocene derivate (metal complex, compound of iron and cyclopentadiene, with chemical characteristics similar to benzene). In the course of the reaction the glucose oxidase enzyme is reduced and during amperometric setting it is again oxidated by ions from ferrocene. The passing flow corresponds to glucose concentration. When using a suitable membrane (for example polyurethane) it is possible to set concentrations up to 50 or mmol.l⁻¹
The solution of heparin and its concentration are measured on potentiometrical basis using the iont—selection electrode in combination with polymer membrane (e.g. polyvinylchloride) with contents of tridodecylmethylamonium chloride as a complex-making agent. The difference of potentials between the reference and measuring electrode corresponds with heparin concentration in blood.
The solution of ethanol and its concentration may be measured similarly to the glucose concentration. The hydrogen peroxide is a product of enzymatic oxidation of glucose or alcohol. An electrode sensitive to hydrogen peroxide contains a membrane with the glucose reductase enzyme. Ethanol diffuses to the enzyme environment and it makes a layer, wherein the ethanol dehydrogenates and hydrogen peroxide is generated. The hydrogen peroxide diffuses to the anode and it produces flow corresponding with the speed of the hydrogen peroxide development.

For each indication solution it is necessary to select a suitable sensor located on the catheter for fast and exact assessment of cardiac output. The precision of the methods does not need repeated performance of measuring and calculations of average results.

The aim of the invention is to design a method of setting the blood flow in bloodstream in a live being body that would simply help—using glucose as an indicator of dilution method of blood flow measuring—to set the blood volume passing through a specific segment of the bloodstream.

### SUMMARY OF THE INVENTION

The invention is based on setting the blood flow through a selected segment of bloodstream using glucose as an indicator of the dilution method.

The above stated aim is reached by setting the blood flow rate in the bloodstream of a live being using glucose as an indicator continuously supplied to the bloodstream and based on the fact that at first, a reference sample is taken in a selected of measured segment of the bloodstream and then, an indicator of known concentration (Gi) and known flow (q) is added to the bloodstream, while—with a time delay—there is performed taking of at least one blood sample from the measuring point, located downstream, while the resulting flow rate through the measured segment is set on the basis of the relation:

$$Q = \frac{q ( Gi - Gs )}{( Gs - Gk )}$$

So as to set the blood flow rate through the bloodstream, it is favourable to perform the blood samples taking with a time delay for the time period of the indicator mixing with the blood.

The resulting flow rate through the measured segment or right atrium of the pulmonary artery is set according to the formula

C(s)=A·C(s)+C₀(s)

It is favourable to perform samples taking intra-corporally or extra corporally.

Device for performance of the method of setting the blood flow through the bloodstream ion a live being body using glucose as an indicator, based on the fact that it contains the indicator tank connected to the linear pump connected with an applicator introduced to the bloodstream, while there is introduced the glucose take-off means connected to the device for setting the glucose concentration and consequently connected to the assessment unit.

The advantage of the method of setting the blood flow through the bloodstream is the fact that there is not needed any special catheter or any special means. The injectomats for indicator supply and catheters are the basic equipment of clinical work sites. The indicator is a common infusion solution and glucose is a body-natural substance.

The organism load with sugar is very low in case of indicator flow of e.g. 0,06 litre per minute (1 ml per second for the period of 20 up to 30 seconds) and it can be tolerated even with diabetics.

Measuring of glucose concentration in taken blood samples (Gk—glucose concentration in blood and Gs—glucose concentration in blood+indicator mixture) can be performed on STATIM basis in bio-chemical laboratory. In the course of handling, the indicator is stable (contrary to chilled liquid). The costs of measuring in the above-described layout are very low.

Compared to dye dilution, there is eliminated the labour-intensive calibration.

The above stated formula for Q calculation has no correction or calibration factor.

Heparin supplies to the patient are not necessary.

There is eliminated the technical problem with blood flow through the measuring chamber (photometry).

There are eliminated mistakes connected with setting the dilution curve area (zero curve line oscillations, recirculation wave interpolation).

Compared to other methods, the glucose measuring by dilution is more precise, as it measures flow of full blood, being a suspension of liquid and solid phase (plasma and erythrocytes). The high precision is supported by significant distance of useful signal from signal noise, as the indicator concentration in the measuring location (Gs) may be more than twice higher in case of the above stated layout of CO measuring compared to the initial value (Gk). The glucose measuring by dilution has high reproductivity.

The process of glucose measuring by dilution is simple compared to other methods and it completes usual measuring of pressures in small circuit.

### LIST OF FIGURES IN DRAWINGS

The presented invention will be explained in detail using a drawing, where

FIG. 1 shows the model scheme of the cardiovascular system sections from the pulmonary artery up to the right ventricle,

FIG. 2 shows comparison of the compliance level of glucose concentration of blood mixture with indicator in a sample: measured-csm and calculated-csv of the first measuring,

FIG. 3 shows comparison of the same level of compliance of csm and csv for the second measuring and

FIG. 4 shows the dilution curve of glucose while measuring the CO in the segment of the right atrium of heart—pulmonary artery,

FIG. 5 shows the measuring chain for setting the blood flow through any segment of the bloodstream,

FIG. 6 shows the measuring chain for setting the cardiac output and cardiac shunt and

FIG. 7 shows the general block scheme of the measuring chain.

### EXEMPLARY EMBODIMENT OF THE INVENTION

The invention will be explained using the example of setting the minute cardiac output (CO=Q) with right-side heart cathetrization with reference to relevant drawings.

There were taken two blood samples for the purpose of setting. These are the following samples. It is the sample for setting the initial (reference) concentration of glucose (Gk) and the sample for setting the glucose concentration in the mixture of both flows (Gs), wherein the mixed blood sample taking is performed at the time when glucose concentration in the place of measuring remains already the same (dilution curve plateau) and it is not affected by recirculation of admixed glucose solution. Indicator supplying is stopped after the blood sample Gs taking. For measuring of minute cardiac output (CO=Q) with right-side heart cathetrization the measuring segment consists of: outlet of the upper or lower vein just in front of the right atrium of heart (place of application) and the pulmonary artery stem (place of measuring). The glucose measuring plateau in the place of measuring is created between the 15^{th} up to 20^{th} second as from the start of the indicator supply to the place of application. The recirculation wave of the indicator used up to the 40^{th} second of its supply does not affect the plateau of Gs concentration. It is possible to take several blood samples from the place of measuring, e.g. every four seconds from the moment of indicator supply start and based on resulting values of blood sugar (Gs1 up to Gsn) it is possible to model the dilution curve course. It is even more favourable—but it is not a condition—to use the possibility of continuous measuring of glucose concentration in the place of measuring, allowing registration of a detailed course of the dilution curve and setting the Gs value in a highly precise way.

Advantages of glucose used as an indicator of the dilution method of blood flow measuring: Glucose is a polar substance, i.e. its molecules unevenly distributed electric charge in the space, creating electric poles by which they may polarize or depolarize each other. Together with glucose, these also include alcohols and water. Polar substances are easily soluble in water (hydrophilic). The molecular weight of glucose is 180,155 g/mol, formula: C₆H₁₂O₆.

The mixing of water solution of glucose in full blood is given by passage of their molecules through the cell membrane of erythrocytes. The passage of substances through cellular membranes is a highly complex operation dependent on physical powers as well as on transport proteins of cellular membranes, concentration, size and electric charge of molecules and other factors as well. It can be divided for example as follows:
Passive transport is mediated by diffusion and osmosis. The water transfer during osmosis is not used in passage of glucose molecules through the cellular membranes. The diffusion, powered by random thermal movement of molecules depending on their different concentration in the solution (free diffusion) as well as through semi-permeable membranes. The direction of molecules movement is set by concentration slope from the location with higher concentration to the location with lower concentration. Via the cellular membranes, there are quickly transported the small molecules, mainly the molecules of non-polar substances (hydrophobic), like O₂, CO₂, N₂, benzene. They quickly diffuse small non-charged polar molecules like water, glycerol or ethanol. Bigger non-charged polar molecules, like amino acids, nucleotides or glucose diffuse slowly and hardly. Transfer of substances by diffusion via the cellular membranes does not require any energy mediated by cellular metabolism.
Active transport of substances through cellular membranes needs some cellular energy and it is released by fission of high-energy relations of the adenosine triphosphate (ATP). An example of “primary active transport” is the sodium—kalium pump which displaces Na⁺ ions out of the cell in exchange of K⁺ ions, taking them towards the cell (3 Na⁺ ions for 2 ions K⁺). But if the molecule or ion of the substance to be taken along during the active transfer, we talk of “secondary active transport”, which may be—depending on the direction of additional substance movement—consonant (contransport, symport) or opposite (antiport). In most of the cells, the glucose molecules are transported via their membranes by symport together with Na⁺ ion.
The transfer of glucose molecules via the cell membrane of erythrocytes is—together with some cells of central nervous system—different than in case of cells of other tissues and organs. It was formerly supposed that the membrane of erythrocytes is freely diffusible for the glucose molecule. At the present time, there are known three classes of transport proteins of cell membrane glucoses marked as GLUT-1 up to GLUT-11, when the glucose transporter of the class I marked GLUT-1 is that one which easies and accelerates diffusion of glucose molecules only via the membrane of erythrocytes, so called facilitated diffusion. Mainly the facilitated diffusion and to some limited level even simple diffusion and symport with ions Na⁺ is the mechanism, which established—after mixing the water solution with glucose—the preassumption that glucose molecules distribute evenly in the whole volume of blood, in plasma suspension and inter cellular space of erythrocytes. So a to check this pre-assumption there were performed two laboratory measuring procedures comparing the resulting glucose concentration in the mixture of blood and glucose solution set by measuring (csm) with calculation (csv), which was performed according to the formula derived from solutions mixing according to their quantity (m) and concentration (c) of investigated substance, a glucose in this case:

csv=(mk*ck+mi*ci)/(mk+mi),

where
csv—concentration of glucose in mixture of blood and added water solution of glucose
ck—concentration of glucose in taken blood sample
ci—concentration of glucose in water solution in mmol/l
mk—full blood volume in ml
mi—volume of added water solution of glucose

The first measuring was performed by manual pipettes. 2 ml (mk) of taken venous blood was completed with 0,02 ml (mi) of 10% glucose solution (10% solution=10 g of the substance is diluted in 100 ml of water=555 mmol/l). The volumes of blood and the solution were performed by exact pipettes, the concentration of glucose in blood and mixture were measured in the biochemical laboratory.

The second measuring was performed with the same parameters, but it was completely performed by an experienced laboratory technician using automatic pipettes.

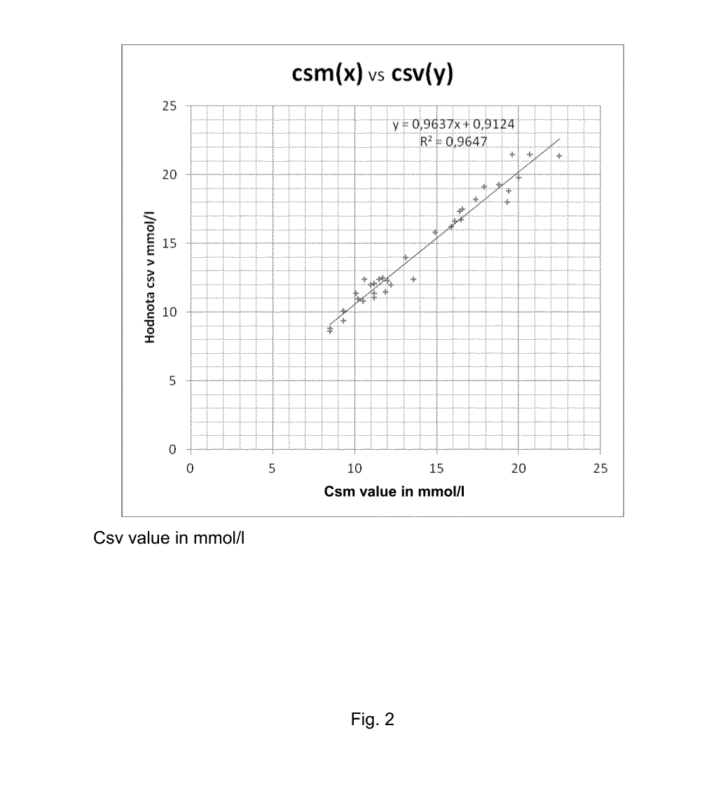

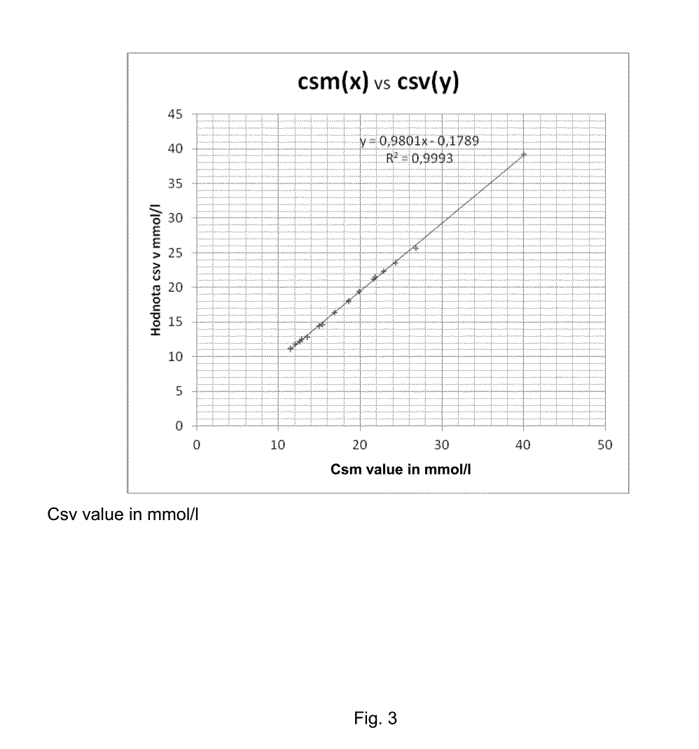

The results of both measuring courses, i.e. comparison of measured concentration of glucose in the mixture of blood and water solution of glucose (csm) with calculation (csv) are shown in the form of a graph in FIG. 2 and FIG. 3. The graphs clearly indicate that compliance of glucose concentration in mixture established by measuring and calculation is high.

The equation for glucose dilution was set in the following way.

This is a modification of a general equation, which was used above, for calculation of mixture of two solutions with different concentrations (k) of the same substance and with their different volumes (m):

c*(m1+m2)=(k1*m1)+(k2*m2), where
c=resulting concentration of mixture of two identical substances
k1 and k2=concentration of the first and the second substance in the solution
m1 and m2=volumes of two solutions, in which there are substance concentrations k1 and k2.

The form of the same equation for mixture of solutions, but using symbols according to the above stated method of cardiac output (CO) measuring using glucose dilution, in which the static volume (m) is replaced with flow (Q, q), is as follows:

Gs*(Q+q)=(Gk*Q)+(Gi*q), where
Gs=concentration of glucose in the mixture of blood and added indicator in mmol/l
Gk=concentration of glucose in blood before supply of indicator in mmol/l
Gi=concentration of glucose in solution of indicator in mmol/l
Q=flow rate of blood in l/min.
q=speed (flow rate) of added solution of glucose (indicator) in l/min.

By adjustment of the equation we get the resulting form for setting the CO (Q):

$$Q = \frac{q ( Gi - Gs )}{( Gs - Gk )}$$

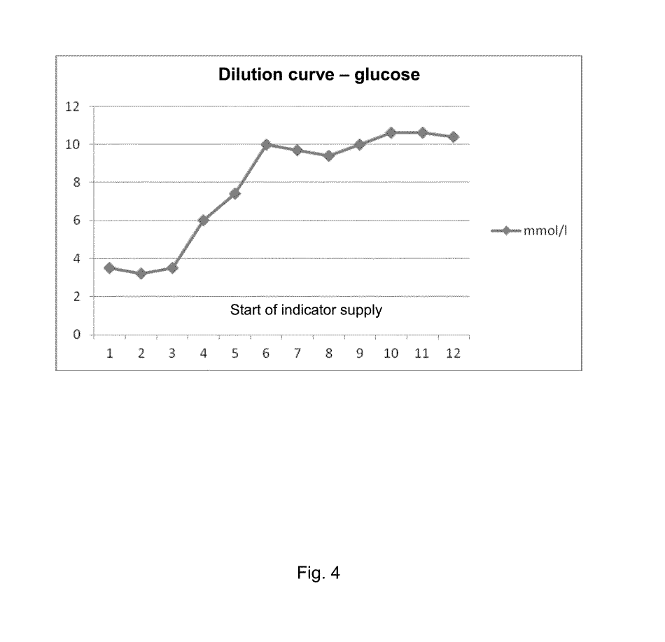

The example of results of cardiac output measuring by glucose dilution according to the invention is shown in FIG. 4. The dilution curve of glucose shows the course of glucose concentration in blood samples taken from the pulmonary artery in four second intervals. Samples No. 1 up to 3 were taken before start of the indicator provision. Provision of 10% solution of glucose in front of the right atrium of heart with the speed of 0,06 1/min. started after taking the sample No. 3.

The average value of samples No. 1 up to 3 is 3,4 mmol/l, the average value of samples No. 9 up to 11 is 10,15 mmol/l, which—based on calculation—corresponds with CO=4,84 l/min.

Thanks to this solution it is possible to set not only minute cardiac output, but also the heart shunt, respectively its exact range. For this purpose, a SW model was developed, allowing the calculation on the basis of the above stated measured data.

When creating the mathematic models, the base is a block scheme where the indicator is injected to the pulmonary artery. The model may be modified to supply of solution to the right atrium of heart. The models (dynamic and static) are created in the calculation environment of the program Matlab & Simulink. The dynamic model is created in the Simulink environment, where there is simulated the course of glucose concentration depending on time while entering all and any prescribed parameters including the cardiac output value and percentual value of left-right shunt. The static model is created in the Matlab environment.

So as to check the models, it is supposed that there is available a patient with suspicion of a LR shunt. The patient is provided with 10% solution of glucose and 2 samples of blood are taken. A dynamic model is created in the Matlab & Simulink program, simulating time courses of glucose concentration in blood of individual compartments. Such a setup model serves mainly for checking the real measured data.

The real contribution and novelty of the presented method according to the invention is included in an inverse task: it is supposed that there are known stable values of glucose concentration at the beginning and in the end of the experiment, obtained by measuring and it is the aim to set the percentual value of the LR shunt. For calculation of the value as well as balanced statuses of all the values in compartments a setup static model is being used as created using the Matlab Symbolic Toolbox tool.

There are calculated the steady states of all the items in the compartments on the basis of Laplace's transformation, to which are transcribed the original integro-differential equations describing the multi-compartment model of the cardiovascular system. An important aim of the models is to obtain a stable value of concentration on the basis of two values (concentration of glucose in blood before and after the solution supply), based on which there is set the percentual value of LR shunt, which is the main and the most important output of the model. The calculations indicate that the LR shunt value may be set on the basis of two concentration values (while knowing the other parameters stated in Table 1).

The calculations and simulations in both mathematical models are based on parameters according to the below stated Table 1 with typical values of an adult man. Table 1 records the volume and time value for each section of the cardiovascular system. The values are different in individual parts of the cardiovascular system.

TABLE 1
Parameters for calculation and simulation

| Marking of | | | | |
|---|---|---|---|---|
| compartments | | Anatomic representation | Volume V [ml] | Time T [s] |
| 0 | (M) | Right ventricle | 125 | 1.25 |
| 1 | (M) | Pulmonary artery | 250 | 2.5 |
| P | (D) | Pulmonary capillaries and | 500 | 5.0 |
| | | veins | | |
| 2 | (M) | Left atrium | 125 | 1.25 |
| 3 | (M) | Left ventricle | 125 | 1.25 |
| 4 | (M) | Aorta, large arteries | 750 | 7.5 |
| 5 | (M) | Small arteries | 200 | 2.0 |
| S | (D) | Capillary system and small | 800 | 8.0 |
| | | veins | | |
| 6 | (M) | Venous system | 1000 | 10 |
| 7 | (M) | Right atrium | 125 | 1.25 |

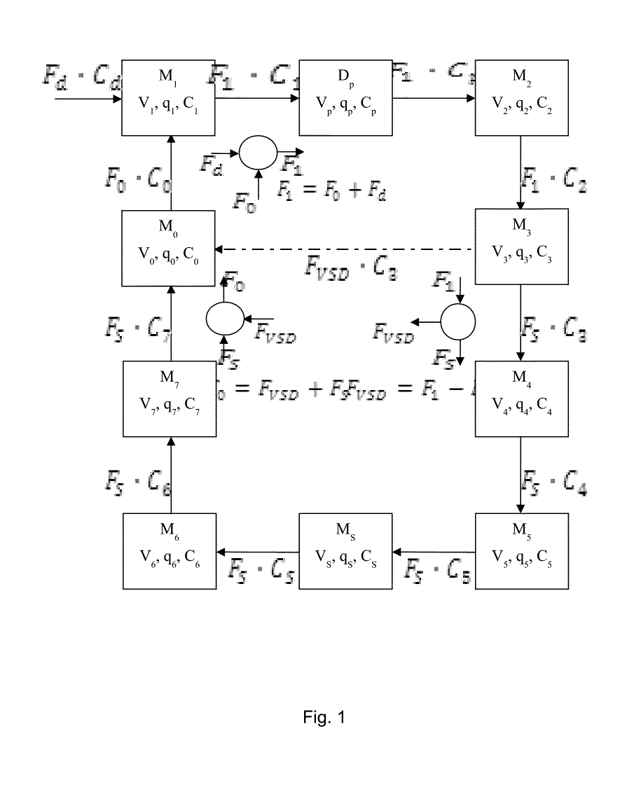

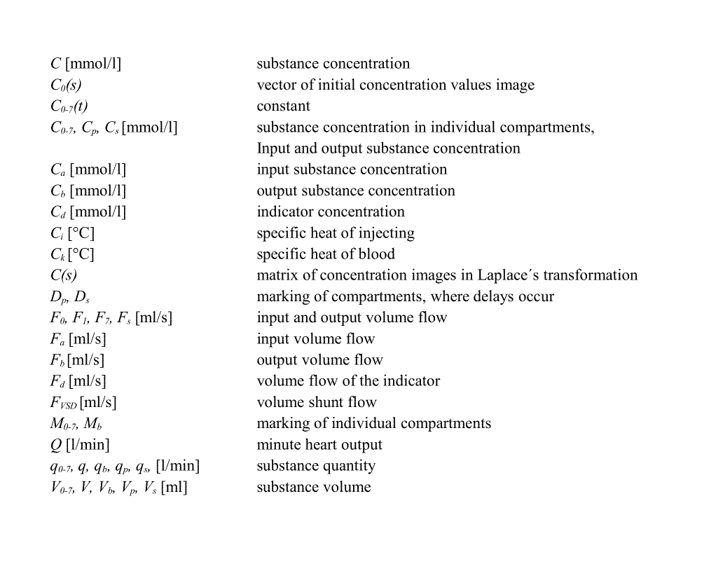

The model is further explained here below using the scheme in FIG. 1. As it can be seen in FIG. 1, the model scheme consists of 10 compartments that gradually represent the individual sections of the cardiovascular system from the pulmonary artery up to the right ventricle, which again connects to the pulmonary artery. In individual compartments it is possible to calculate the value of glucose concentration in blood. The scheme depicts the combination of time delay and perfect mixing of the indicator with blood in individual compartments. There is indicated the place of indicator application to the pulmonary artery as well as the left-right shunt when the blood divides in the left ventricle—part of the blood goes to aorta and part of the blood flows to the right ventricle.

Some time delay occurs in compartments Dp and Ds. The first compartment represents the delay in the pulmonary system, the second compartment represents the delay in the general system.

In other compartments, the dilution curves are indicated and they show the concentration depending on time. This model is used for development of a mathematic model in Simulink exactly for drawing the individual dilution curves, where it will also be possible to simulate the left-right shunt. The values of glucose concentration are expressed on the basis of integro-differential equations.

The compartment M0 represents the right ventricle and there flows the shunt volume from the left ventricle. The glucose concentration is expressed as follows:

$${C}_{0} ( t ) = \frac{1}{{V}_{0}} {\int }_{0}^{t} ( {F}_{S} {C}_{7} + {F}_{VSD} {F}_{3} - {F}_{0} {C}_{0} ) d t + {C}_{0} ( 0 ) \qquad ( 8.2 )$$

where

| | |
|---|---|
| C₀₋₇(t) | invariable |
| V₀₋₇, V, V_{b}, Vₚ, Vₛ [ml] | substance volume |
| F₀, F₁, F₇, Fₛ [ml/s] | input and output volume flow rate |
| F_{VSD} [ml/s] | volume shunt flow rate |

The compartment M1 represents the pulmonary artery. There is applied the glucose solution, with its concentration being expressed as follows:

$${C}_{1} ( t ) = \frac{1}{{V}_{1}} {\int }_{0}^{t} ( {F}_{0} {C}_{0} - {F}_{1} {C}_{1} + {F}_{d} {C}_{d} ) d t + {C}_{1} ( 0 ) \qquad ( 8.3 )$$

where

| | |
|---|---|
| C₀₋₇(t) | invariable |
| V₀₋₇, V, V_{b}, Vₚ, Vₛ [ml] | substance volume |
| F₀, F₁, F₇, Fₛ [ml/s] | input and output volume flow rate |
| F_{d} [ml/s] | volume flow rate of indicator |
| C_{d} [mmol/l] | indicator concentration |

The compartment Dp represents the pulmonary capillaries and veins. The glucose concentration is expressed as follows:

Cₚ(t)=C₁(t−Tₚ) (8.4)

where

| | |
|---|---|
| C₀₋₇, Cₚ, Cₛ [mmol/l] | substance concentration in |
| | individual compartments, |
| C₀₋₇(t) | invariable |
| Tp [t] | delay in pulmonary system |

The compartment M2 represents the left atrium of heart. The glucose concentration is expressed as follows:

$${C}_{2} ( t ) = \frac{1}{{V}_{2}} {\int }_{0}^{t} ( {F}_{1} {C}_{p} - {F}_{1} {C}_{2} ) d t + {C}_{2} ( 0 ) \qquad ( 8.5 )$$

where

| | |
|---|---|
| C₀₋₇(t) | invariable |
| V₀₋₇, V, V_{b}, Vₚ, Vₛ [ml] | substance volume |
| F₀, F₁, F₇, Fₛ [ml/s] | input and output volume flow rate |
| C₀₋₇, Cₚ, Cₛ [mmol/l] | substance concentration in |
| | individual compartments, |
| | input and output substance |
| | concentration |

The compartment M3 represents the left ventricle. The shunt defect occurs here. The glucose concentration is expressed as follows:

$${C}_{3} ( t ) = \frac{1}{{V}_{3}} {\int }_{0}^{t} ( {F}_{1} {C}_{2} - {F}_{VSD} {C}_{3} + {F}_{S} {C}_{3} ) d t + {C}_{3} ( 0 ) \qquad ( 8.6 )$$

where

| | |
|---|---|
| C₀₋₇(t) | invariable |
| V₀₋₇, V, V_{b}, Vₚ, Vₛ [ml] | substance volume |
| F₀, F₁, F₇, Fₛ [ml/s] | input and output volume flow rate |
| F_{VSD} [ml/s] | volume shunt flow rate |

The compartment M4 represents the aorta (heart artery) and the glucose concentration is expressed as follows:

$${C}_{4} ( t ) = \frac{1}{{V}_{4}} {\int }_{0}^{t} ( {F}_{S} {C}_{3} - {F}_{S} {C}_{4} ) d t + {C}_{4} ( 0 ) \qquad ( 8.7 )$$

where

| | |
|---|---|
| C₀₋₇(t) | invariable |
| V₀₋₇, V, V_{b}, Vₚ, Vₛ [ml] | substance volume |
| F₀, F₁, F₇, Fₛ [ml/s] | input and output volume flow rate |

The compartment M5 represents small arteries and the glucose concentration is expressed as follows:

$${C}_{5} ( t ) = \frac{1}{{V}_{5}} {\int }_{0}^{t} ( {F}_{S} {C}_{4} - {F}_{5} {C}_{5} ) d t + {C}_{5} ( 0 ) \qquad ( 8.8 )$$

where

| | |
|---|---|
| C₀₋₇(t) | invariable |
| C₀₋₇, Cₚ, Cₛ [mmol/l] | substance concentration in |
| | individual compartments, input |
| | and output substance concentration |
| V₀₋₇, V, V_{b}, Vₚ, Vₛ [ml] | substance volume |
| F₀, F₁, F₇, Fₛ [ml/s] | input and output volume flow rate |

The compartment Ds represents the capillary and venous system. The glucose concentration is expressed as follows:

Cₛ(t)=Cₛ(t−Tₛ) (8.9)

where

| | |
|---|---|
| C₀₋₇, Cₚ, Cₛ [mmol/l] | substance concentration in |
| | individual compartments, input |
| | and output substance concentration |
| Ts [s] | delay in general system |

The compartment M6 represents the venous and the glucose concentration is expressed as follows:

$${C}_{6} ( t ) = \frac{1}{{V}_{6}} {\int }_{0}^{z} ( {F}_{S} {C}_{S} - {F}_{S} {C}_{6} ) d t + {C}_{5} ( 0 ) \qquad ( 8.10 )$$

where

| | |
|---|---|
| C₀₋₇(t) | invariable |
| C₀₋₇, Cₚ, Cₛ [mmol/l] | substance concentration in |
| | individual compartments, input |
| | and output substance concentration |
| V₀₋₇, V, V_{b}, Vₚ, Vₛ [ml] | substance volume |
| F₀, F₁, F₇, Fₛ [ml/s] | input and output volume flow rate |

The compartment M7 represents the right atrium of heart and the concentration is expressed as follows:

$${C}_{7} ( t ) = \frac{1}{{V}_{7}} {\int }_{0}^{z} ( {F}_{5} {C}_{6} - {F}_{5} {C}_{7} ) d t + {C}_{5} ( 0 ) \qquad ( 8.11 )$$

where

| | |
|---|---|
| C₀₋₇(t) | invariable |
| C₀₋₇, Cₚ, Cₛ [mmol/l] | substance concentration in |
| | individual compartments, input |
| | and output substance concentration |
| V₀₋₇, V, V_{b}, Vₚ, Vₛ [ml] | substance volume |
| F₀, F₁, F₇, Fₛ [ml/s] | input and output volume flow rate |

The scheme shows the method of mixing of various volume flows in specific parts of the model, i.e. in the pulmonary artery, left ventricle and right ventricle.

The volume flow rate F1 outgoing from the compartment M1 is expressed by a sum of the volume flow rate Fd of the indicator and the volume flow rate F0, outgoing from the compartment M0.

F₁=F_{d}+F₀ (8.12)

where

| | |
|---|---|
| F₀, F₁, F₇, Fₛ [ml/s] | input and output volume flow rate |
| Fd [ml/s] | volume flow rate of indicator |

The volume flow rate of the shunt FVSD outgoing from the compartment M3 towards the right ventricle is expressed by the difference between the volume flow rate F1 outgoing from the compartment M2 and the volume flow rate FS, outgoing from the compartment M3 and continuing to the compartment M4.

F_{VSD}=F₁−F_{S} (8.13)

where

| | |
|---|---|
| F_{VSD} [ml/s] | volume shunt flow rate |
| F₀, F₁, F₇, Fₛ [ml/s] | input and output volume flow rate |

The volume flow rate F0 outgoing from the compartment M0 is expressed by a sum of the volume flow rate FS outgoing from the compartment M7 and volume shunt flow rate FVSD outgoing from the compartment M3.

F₀=F_{S}+F_{VSD} (8.14)

where

| | |
|---|---|
| F_{VSD} [ml/s] | volume shunt flow rate |
| F₀, F₁, F₇, Fₛ [ml/s] | input and output volume flow rate |

The values of stable statuses of indicator concentration in individual compartments are calculated on the basis of the following relation, when the proper calculation is performed based on a code (Appendix 2—Script of static model for drawing of stable statuses, calculation of stable concentration value and calculation of percentual value of a shunt in case of application to the pulmonary artery) in the program Matlab&Simulink. For the calculation purposes, the code contains the values from table 1. The relation includes the individual matrices as stated here. On the basis of the given relation in the matrix it is possible to calculate the stable value of concentration based on the fact that there is known the percentual value of the shunt. It is also possible to calculate the percentual value of the shunt on the basis of the fact that there is known the stable value of concentration.

The calculation will be performed on the basis of the following equation

C(s)=A·C(s)+Cₚ(s) (8.15)

where: C(s) represents the matrix of concentration images in Laplace's transformation
A is an auxiliary matrix
C₀(s) is the vector of images of initial values of concentrations.

The above stated equation represents a calculation performed with the use of matrices that are as follows:

$$A = ( \begin{matrix}- \frac{{F}_{0} ( s )}{{V}_{1} s} & 0 & 0 & 0 & 0 & 0 & 0 & 0 & 0 & \frac{{F}_{0} ( S )}{{V}_{1} S} \\ {e}^{- {r}_{s} s} & 0 & 0 & 0 & 0 & 0 & 0 & 0 & 0 & 0 \\ 0 & \frac{{F}_{0} ( s )}{{V}_{2} s} & - \frac{{F}_{0} ( s )}{{V}_{2} s} & 0 & 0 & 0 & 0 & 0 & 0 & 0 \\ 0 & 0 & \frac{{F}_{0} ( s )}{{V}_{3} s} & \frac{- {F}_{VSD} ( S ) - {F}_{S} ( S )}{ {V}_{2} S} & 0 & 0 & 0 & 0 & 0 & 0 \\ 0 & 0 & 0 & \frac{{F}_{S} ( S )}{{V}_{4} S} & - \frac{{F}_{S} ( S )}{{V}_{4} S} & 0 & 0 & 0 & 0 & 0 \\ 0 & 0 & 0 & 0 & \frac{{F}_{s} ( S )}{{V}_{3} S} & - \frac{{F}_{s} ( S )}{{V}_{3} S} & 0 & 0 & 0 & 0 \\ 0 & 0 & 0 & 0 & 0 & {e}^{- {r}_{s} S} & 0 & 0 & 0 & 0 \\ 0 & 0 & 0 & 0 & 0 & 0 & \frac{{F}_{s} ( S )}{{V}_{4} S} & - \frac{{F}_{s} ( S )}{{V}_{6} S} & 0 & 0 \\ 0 & 0 & 0 & 0 & 0 & 0 & 0 & \frac{{F}_{S} ( S )}{{V}_{7} S} & - \frac{{F}_{7} ( s )}{{V}_{7} s} & 0 \\ 0 & 0 & 0 & \frac{{F}_{VSD}}{{V}_{0} S} & 0 & 0 & 0 & 0 & \frac{{F}_{7} ( s )}{{V}_{0} s} & - \frac{{F}_{0} ( S )}{{V}_{0} S}\end{matrix} )$$

$$C ( s ) = ( \begin{matrix}{C}_{1} ( s ) \\ {C}_{p} ( s ) \\ {C}_{2} ( s ) \\ {C}_{3} ( s ) \\ {C}_{4} ( s ) \\ {C}_{5} ( s ) \\ {C}_{s} ( s ) \\ {C}_{6} ( s ) \\ {C}_{7} ( s ) \\ {C}_{0} ( s )\end{matrix} ) {C}_{0} ( s ) = ( \begin{matrix}{C}_{10} ( s ) \\ {C}_{p 0} ( s ) \\ {C}_{20} ( s ) \\ {C}_{30} ( s ) \\ {C}_{40} ( s ) \\ {C}_{50} ( s ) \\ {C}_{s 0} ( s ) \\ {C}_{60} ( s ) \\ \frac{U ( s )}{{V}_{1} s} + {C}_{70} ( s ) \\ {C}_{00} ( s )\end{matrix} )$$

Device for setting the cardiac output which is used for application of an indicator of known concentration, in this case it is glucose. The device arranges continuous dosage of the indicator—in this case glucose—to the bloodstream and consequent taking of samples and their analysis. On their basis it is possible to set the cardiac output and in case of application performance in the close vicinity of heart, it is also possible to detect a heart shunt and its extent.

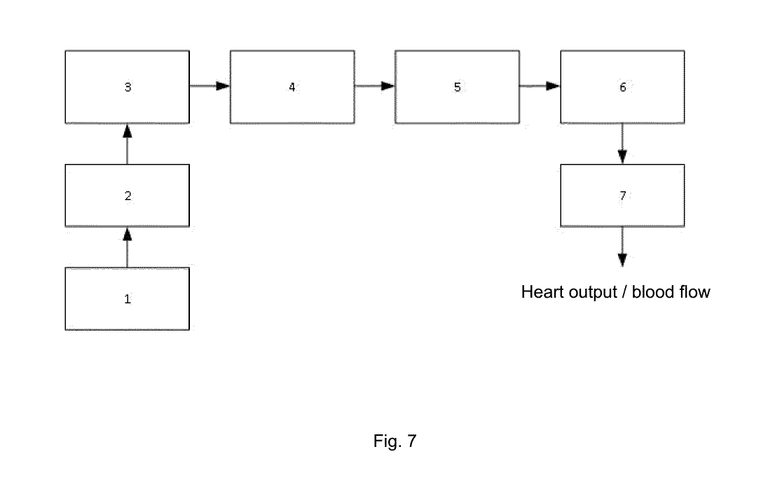

The general scheme in case of establishing the blood flow rate in any part of the bloodstream between the indicator application and sample taking, without branching—it is shown in FIG. 7. In this case the measuring chain includes indicator 1 with known concentration Gi is applied—using the linear pump 2 with constant known speed of dosage—to the spot of application A by the speed q. In the bloodstream 3, with blood flowing with the speed Q, the blood is mixed with glucose concentration Gk with the indicator 1 of known concentration Gi and so there develops a mixture of blood and indicator 1 with general concentration Gs. Using the taking means 4 for example a syringe there are taken at least two samples of blood with the volume V. The first sample is taken before application of the indicator 1 to the bloodstream 3. And at least one another sample is taken from the same place in time t, wherein the other samples may be taken in times t+1, t+2, etc. Based on these taken samples there is established the concentration of glucose in device 5 for setting the glucose concentration and consequently the assessment unit 6 is used for algorithm-based setting of the blood flow rate through the given segment of the bloodstream 3.
Indicator 1—there is used the solution of glucose of known concentration. It is possible to use several variants. With higher glucose concentration there will be lower speed of the indicator supply.
Linear pump 2—standard infusion pump allowing supply of the indicator 1 by required speed. Instead of the pump it is possible to use a syringe with “pseudo” constant supply of the indicator.
Bloodstream 3—it represents any part of the cardiovascular system.
Taking means 4—it may be a syringe or a catheter introduced to the spot of taking and used for transfer of blood from the taking spot to the syringe or a catheter.
Device 5 for setting the glucose concentration—it may be a laboratory appliance for examination of blood samples or a special catheter with a sensor for on-line setting of glucose concentration in blood.
Assessment unit 6—a device containing an algorithm for setting the cardiac output. In case of measuring in the close vicinity of heart even an algorithm for heart shunt setting, including its range.
Program application 7

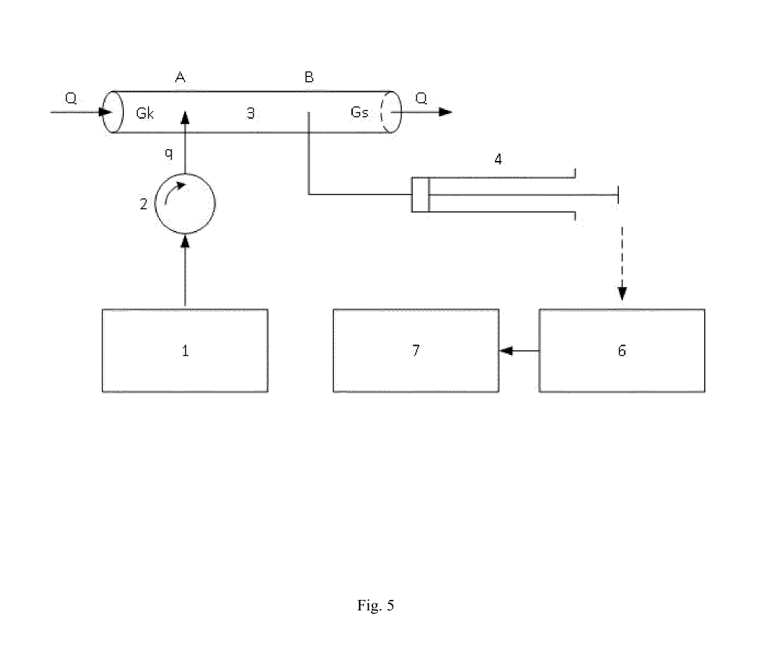

FIG. 5 shows a simplified scheme of the measuring chain in case of establishing the blood flow rate in any part of the bloodstream, which does not branch between the indicator application and sample taking. The indicator 1 with known concentration Gi is applied using the linear pump 2 with constant known speed of dosage q to the spot of application A by the speed q. In the bloodstream 3, where blood flows with the speed Q, the blood is mixed with glucose concentration Gk with the indicator 1 of known concentration Gi and so there develops a mixture of blood and indicator 1 with general concentration Gs. Using the taking syringe there are taken at least two samples of blood with the volume V. The first sample is taken before application of the indicator 1. And at least one another sample is taken from the same place in time t. Based on these taken samples there is established the concentration of glucose and consequently there is used an algorithm for setting the blood flow rate through the given segment of the bloodstream 3. The indicator 1 may be supplied by a linear pump 2, e.g. by an infusion pump, linear dosing device, which guarantees “constant” speed of supply. The proper applicator may be any cannula, infusion set, introduced to the bloodstream in spot A.

Possible variants of sample taking:
Sample taking is again possible using the syringe of any volume.
Taking can also be performed using a designed catheter. It depends on the taking location. If we take blood from a vein on a limb which is easily accessible, a syringe is sufficient. In case of taking from the heart, a catheter is needed (a suitable one is e.g. the Swan-Ganz catheter) with two inputs—one of them may be used for the indicator application, the other one may be used for sample taking from the same place.

Possible variants of setting the glucose in blood:

Laboratory setting of glucose concentration—the sample is investigated in a biochemical laboratory using a suitable investigation method and an exact glucose concentration in blood is set.

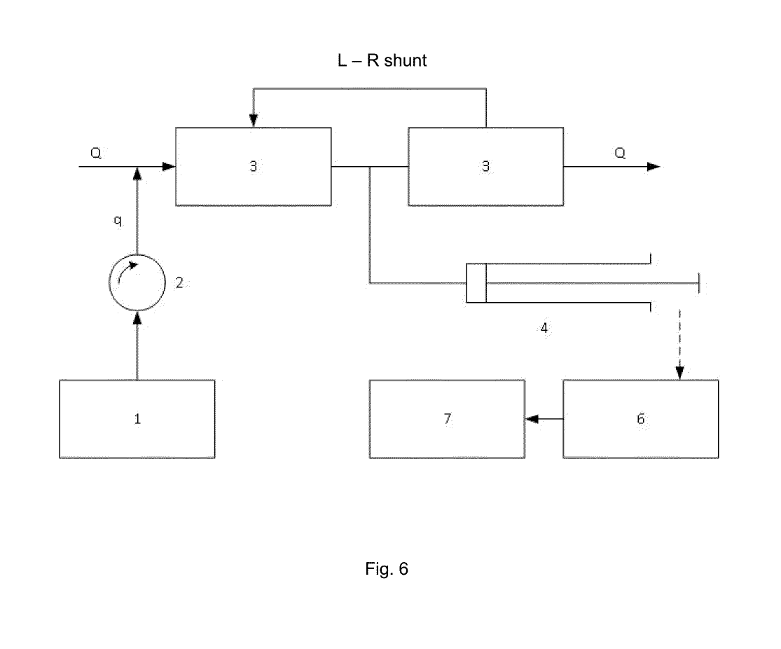

Online glucose concentration setting
The sensor is placed at the catheter tip, i.e. directly in the bloodstream
   this method is not being used.
The sensor is placed out of the catheter body, working on the micro-dialyse principle
FIG. 6 shows a similar measuring chain, but for the case of cardiac output setting, i.e. for the indicator application in the close vicinity of heart, respectively at the entry to the heart, i.e. upper or lower vena cava and the place of blood samples taking is the outlet of the right ventricle, i.e. ideally the beginning of the pulmonary artery with marking of left-right intra-heart shunt.

Possible variants of indicator application:
Even in this case it is possible to use both of the above stated variants of the indicator application, but in case of using the manual application by a syringe it is necessary to consider application via any catheter or a hollow hose.

Possible variants of sample taking:
Sample taking must also be done through a catheter and then using a syringe.

Possible variants of setting the glucose in blood:
Everything is identical as in case of the above stated measuring chain.

### LIST OF REFERENCE NUMBERS

1 indicator
2 linear pump
3 bloodstream
4 taking means
5 device for setting the indicator/glucose concentration
6 assessment unit
7 program application for setting the blood flow
8 indicator tank
9 indicator applicator

APPENDIX 2
clear all,close all,clc
tic
%ustaleny stav
syms s Tp Ts Vp Vs V1 V2 V3 V4 V5 V6 V7 V0 F0 F1 Fvsd Fs Fd LFd
LFdCd
syms Cp0 Cs0 C10 C20 C30 C40 C50 C60 C70 C00
syms Cp C0 C1 C2 C3 C4 C5 C6 C7
%%%%%%%%%%%%%%%%%%%% zadani %%%%%%%%%%%%
%%%%%%%%%%%%%%%%
Fd_konst=1;t1=80;t2=104; %zaciname vstrikovat v case 80
sekund, vstrikavame po dobu 24 sekundy roztok 10 procent
glukozy, tj. 555
roztok_koncentrace=555;
Fd_value=Fd_konst*(1/s*exp(−t1*s)−1/s*exp(−t2*s));
Cpoc=5; % pocatecni hodnota koncentrace
U=roztok_koncentrace*Fd_value;
x0=[C10;C20;C30;C40;C50;C60;C70;C00];
C10_value=Cpoc;
C20_value=Cpoc;
C30_value=Cpoc;
C40_value=Cpoc;
C50_value=Cpoc;
C60_value=Cpoc;
C70_value=Cpoc;
C00_value=Cpoc;
Cp0_value=0;
Cs0_value=0;
% hodnoty p\check{r}ebrané z tabulky 1
V0_value=125;
V1_value=250;
V2_value=125;
V3_value=125;
V4_value=750;
V5_value=200;
V6_value=1000;
V7_value=125;
Vp_value=500;
Vs_value=800;
srdecni_vydej=100; %zadani srdecniho vydeje
VSD_procenta=20; %hodnota zkratu v procentech z prutoku F1
(srdecniho vydeje)
%%%%%%%%%%%%%%%%%%%%%%%% zadani %%%%%%%%
%%%%%%%%%%%%%%%%
F1_value=srdecni_vydej/s; %obraz hodnoty srdecniho vydeje
Fvsd_value=VSD_procenta/100*F1_value; %obraz zkratu, aktualne
20% ze 100,tj 20
F0_value=F1_value−Fd_value;
Fs_value=F1_value−Fvsd_value;
Tp=Vp/F1;
Ts=Vs/F1;
A=[−F1/V1/s 0 0 0 0 0 0 0 0 F0/V1/s;...

exp(−Tp*s) 0 0 0 0 0 0 0 0 0;...
0 F1/V2/s −F1/V2/s 0 0 0 0 0 0 0;...
0 0 F1/V3/s (−Fvsd−Fs)/V3/s 0 0 0 0 0 0;...
0 0 0 Fs/V4/s −Fs/V4/s 0 0 0 0 0;...
0 0 0 0 Fs/V5/s −Fs/V5/s 0 0 0 0;...
0 0 0 0 0 exp(−Ts*s) 0 0 0 0;...
0 0 0 0 0 0 Fs/V6/s −Fs/V6/s 0 0;...
0 0 0 0 0 0 0 Fs/V7/s −Fs/V7/s 0;...
0 0 0 Fvsd/V0/s 0 0 0 0 Fs/V0/s −F0/V0/s];

b=[U/V1/s+C10;Cp0;C20;C30;C40;C50;Cs0;C60;C70;C00];
I=eye(size(A));
C=inv(I−A)*b;
c_ust_pokus_bezFvsd=subs(C,
{Cs0,Cp0,Vp,Vs,Fs,F1,F0,C10,C20,C30,C40,C50,C60,C70,C00,V0,V1,
V2,V3,V4,V5,V6,V7},
{Cs0_value/s,Cp0_value/s,Vp_value,Vs_value,Fs_value,F1_value,F
0_value,C10_value/s,C20_value/s,C30_value/s,C40_value/s,C50_va
lue/s,C60_value/s,C70_value/s,C00_value/s,V0_value,V1_value,V2
_value,V3_value,V4_value,V5_value,V6_value,V7_value});
%ZNAM ZKRAT, CHCI SPOCIST USTALENOU HODNOTU
c_ust=subs(c_ust_pokus_bezFvsd,Fvsd,Fvsd_value);
c_ust_value=limit(s*c_ust,s,0);
vpa(c_ust_value,7)
%ZNAM USTALENOU HODNOTU, CHCI SPOCIST ZKRAT
ustalena_koncentrace=6.9844; % hodnota se zada z vypo\check{c}tené
ustálené hodnoty
syms ust
zkrat_obraz=solve(c_ust_pokus_bezFvsd(1)−ust/s,Fvsd);
ustalena_hodnota_sym=limit(s*zkrat_obraz,s,0);
ezplot(ustalena_hodnota_sym,[5.8 10.8]),xlabel(‘ustálená
koncentrace [mmol/l]’),ylabel(‘zkrat
[%]’),grid,title(‘Závislost koncentrace na zkratu’)
zkrat=subs(ustalena_hodnota_sym,ust,ustalena_koncentrace)
toc

1. A method of estimating a blood flow in a bloodstream in a live being body using glucose as an indicator continuously supplied to the bloodstream, the method comprising:
taking a reference sample in a selected location of a measured segment of the bloodstream;
thereafter, adding an indicator of known concentration (Gi) and known flow (q) to the bloodstream;
after some time interval, taking at least one blood sample from a downstream measuring location; and
arriving at an estimate of the resulting flow through the measured segment according to the following formula
$$Q = \frac{q ( Gi - Gs )}{( Gs - Gk )}$$

2. The method of claim 1, characterised in that the taking of blood samples is performed in a time delay for a time of indicator mixing with the blood.

3. The method of claim 1, characterised in that the resulting flow through a measured segment of a right atrium of a pulmonary artery is estimated based on the relation
C(s)=A·C(s)+C₀(s)

4. The method of claim 2, characterised in that a resulting flow through a measured segment of a right atrium of a pulmonary artery is estimated based on the relation
C(s)=A·C(s)+C₀(s)

5. The method of estimating the blood flow in the bloodstream in a live being body using glucose as an indicator according to claim 1, characterised in that that samples taking and setting the glucose concentration in blood is performed intra-corporally or extra-corporally.

6. The method of estimating the blood flow in the bloodstream in a live being body using glucose as an indicator according to claim 2, characterised in that that samples taking and setting the glucose concentration in blood is performed intra-corporally or extra-corporally.

7. The method of estimating the blood flow in the bloodstream in a live being body using glucose as an indicator according to claim 3, characterised in that that samples taking and setting the glucose concentration in blood is performed intra-corporally or extra-corporally.

8. A device for performing the method of estimating the blood flow in the bloodstream in a live being body using glucose as an indicator according to claim 1, characterised in that that it includes a reservoir (8) of an indicator (1) connected to a linear pump (2) connected with an applicator (9) introduced to the bloodstream (3) to which is connected a drawing means (4) of glucose, connected to a device (5) for setting the concentration of glucose and consequently in an assessment unit (6).
